(19) United States
(12) Patent Application Publication
(10) Pub. No.: US 2023/0181398 A1
(43) Pub. Date: Jun. 15, 2023

(54) DEVICE FOR LIMITING INFECTION

(71) Applicant: CREATIVE HINGE PTY LTD., Mosman, New South Wales (AU)

(72) Inventors: John LEYDEN, Mosman, New South Wales (AU); Les HILL, Mosman, New South Wales (AU)

(73) Assignee: CREATIVE HINGE PTY LTD., Mosman, New South Wales (AU)

(21) Appl. No.: 17/916,556

(22) PCT Filed: Apr. 1, 2021
(86) PCT No.: PCT/AU2021/050305

§ 371 Date: Sep. 30, 2022

(30) Foreign Application Priority Data
Apr. 1, 2020 (AU) 2020901004

(51) Int. Cl. A61G 10/00; A61G 10/02; A61M 11/02; A61M 16/00

(52) U.S. Cl. CPC A61G 10/005; A61G 10/02; A61M 11/02; A61M 16/0087

(57) ABSTRACT

Disclosed is a barrier tent comprising a canopy, at least a portion of the canopy being composed of flexible sheet material; a frame for supporting the canopy; the canopy and frame being configured such that an internal surface of the canopy defines at least a portion of a patient segregation cavity to enclose a patient's head and at least a portion of the patient's body within the patient segregation cavity; and a gas input extending through the canopy, the gas input being positioned at a location configured such that supply of a gas through the input limits movement of aerosolised particles generated by or adjacent the patient toward the internal surface of the canopy.

### BACKGROUND

In the setting of viral and bacterial spread of highly communicable disease such as COVID-19, concerns about increased rates (incidence) and severity of infections in healthcare workers are prominent in the health industry, creating fear, anxiety and the real risk of becoming ill or dying whilst providing care to patients.

Current estimates of healthcare workers being infected during the current COVID-19 pandemic range from 9-14% of all infections which is less than the 21% that occurred during the SARS outbreak in 2002, however, we are in the early phase of the current pandemic.

The greatest risk of infection to healthcare workers occurs during any aerosol producing intervention or treatment where the concentration of infective airborne particles is increased, in close proximity to the attending medical or allied person. This larger concentration of particles can produce a more severe infection in the attending medical personal due to the larger inoculation dose of virus particularly in the upper airways.

Current precautions to minimise infection include:
Use of standard precautions—hand hygiene, cough etiquette, universal precautions
Use of contact and droplet precautions—surgical mask, eye shield/goggle protection, long sleeved gowns, gloves.
Use of contact and airborne precautions—P2 respirator, eye shield/goggle protection, imperviable gown, gloves

The aerosol generating procedures that are of most concern include:
Non-invasive ventilation (simple mask, CPAP or BiPAP)
Bag and mask ventilation
Endotracheal intubation
Ventilation via supraglottic airways (eg laryngeal mask) including their insertion and removal
High flow nasal oxygen therapy
Diagnostic and therapeutic instrumentation of the upper airway and oropharynx including bronchoscopy, tracheostomy, upper gastrointestinal endoscopy
Cardiopulmonary resuscitation
Any surgical procedure whereby aerosolizaton of tissue may occur.

For decades, the medical industry has used prevention, blocking or shielding systems to mitigate exposure to aerosolised infective particles, such items include gowns, gloves, masks, hoods, respirators, shields, goggles and methods of physical separation.

### SUMMARY OF DISCLOSURE

Disclosed is a barrier tent comprising a canopy, at least a portion of the canopy being composed of flexible sheet material; a frame for supporting the canopy; the canopy and frame being configured such that an internal surface of the canopy defines at least a portion of a patient segregation cavity to enclose a patient's head and at least a portion of the patient's body within the patient segregation cavity; and a gas input extending through the canopy, the gas input being positioned at a location configured such that supply of a gas through the input limits movement of aerosolised particles generated by or adjacent the patient toward the internal surface of the canopy.

In some forms the gas input is located at an apex or upper portion of the segregation cavity.

The barrier tent acts as a mobile safety barrier to protect health workers and other essential services from infection through any aerosol producing event, treatment or action. The barrier tent has the benefits of simple storage and quick deployment for use. Deployment automatically positions the gas input in the correct location to limit an aerosol from the patient contacting the interior of the tent. This limits the danger of infection of the health worker or user.

In some forms the frame comprises a plurality of flexible elongate members allowing for a flexible and easily constructed safety barrier.

In some forms the barrier tent is configured such that supply of a gas through the input disperses gas downwards within the segregation cavity in a conical spray pattern. This spray pattern limits the movement of aerosol to the interior surfaces of the tent.

In some forms the barrier tent further comprises one or more semi-sealed apertures extending through the cavity to allow access to the patient. This allows a health worker to perform procedures and access the head of the patient while maintaining safety.

In some forms the barrier tent has suction assisted removal of gases within the segregated patient cavity.

The current invention does not lessen the applicability or utility of existing systems but additionally and significantly limits aerosol dispersion during specific interventions by use of a space-limiting impermeable barrier tent.

The barrier tent may be utilised under various conditions where there is risk of aerosolised dispersion of viral, bacterial or particulate material from a patient into the immediate surrounds. Containment of such material is imperative to minimise subsequent infection or contamination of persons in close proximity.

The barrier tent of the disclosure provides a physical barrier protection against bacteria, viruses, foreign material (eg blood, sputum or other bodily fluid/tissue) aerosolised by an infected person (eg normal breathing, coughing, sneezing etc) or by an intervention. The material of the tent is designed not to be permeated by the aerosolised material. In some forms the material may be a polymer or other impermeable sheet material. In some forms a portion of the tent may be rigid while other portions are flexible,

In some forms, the barrier tent provides respiratory support in the form of an oxygen tent, for example.

Interventions may occur within the confines of the tent whilst providing barrier protection to the interventionist. For example, intervention may include endotracheal intubation, bronchoscopy or other procedures. Access to the confines of the tent for such interventions may occur via semi-sealed access ports. In some forms those ports may be in the form of apertures such as circular apertures with a split cover. In this form the apertures allow a user to insert an arm into the confines of the tent without opening the protection area. In some forms those ports may be in the form of flap access through the impermeable tent curtain.

In some forms gas input into the tent impedes the flow of aerosolised particles generated by procedures. In some forms the gas input is configured to promote movement of aerosolised particles away from the internal tent surface by use of full cone spray nozzles located at or proximal to the tent apex. Those spray nozzles or other gas inputs disperse gas flow downwards, in some forms in a conical spray pattern. In some forms the shape of the pattern or the positioning of the input is designed to allow for directing aerosolised material emanating from or near the patient to be directed away from the sides of the tent.

In particular, the apertures or ports in the tent are located in a position to be away from the direction of the aerosolised material. In addition, the apertures may be oriented such that the opening faces away from the flow of aerosolised particles. For example, the apertures may be positioned toward an upper portion of the interior of the cavity while the aerosolised material is directed downwardly toward the lower portion of the interior cavity. This limits the possibility of infection.

In some forms the tent allows for suction assisted removal of gases. In some forms suction elements are located at the base of the tent and may be in the form of flat perforated suction strips or perforated tubes or other suction elements.

In some forms, adhesive strips located at the base of the tent form a seal against bedding material or bed sides.

Overall the device may have the benefit of providing a compact, free-standing, non-sterile tent that is easily erected to create a covered space around the head and upper torso of a person supine or semi-recumbent on a trolley or a bed. The physical aspect of the tent and the application of low-pressure suction to the basal extraction system creates a negative pressure environment which contains and extracts a significant concentration of particles within the tent thereby limiting spread of particles in the environment. The addition of apertures to the barrier walls allows access to the interior for manipulations whilst minimising escape of particles to the exterior. The tent is a durable, single use device, that is an addition to current and future protective processes or systems to minimise the spread of particles between persons during active interventions or ambient conditions.

### BRIEF DESCRIPTION OF THE FIGURES

FIG. 1 shows an isometric view of one form of the tent barrier of the present disclosure in use;

FIG. 2 shows an isometric view of a second embodiment of the tent barrier;

FIG. 3 shows an isometric view of a third embodiment of the tent barrier in use;

FIG. 4 shows an upper view of a tent barrier of FIG. 3 and a close up view of the gas input of the third embodiment of the disclosure;

FIG. 5 shows an isometric view of a fourth embodiment of the tent barrier in use;

FIG. 6 shows an isometric view of a fifth embodiment of the tent barrier in use;

FIG. 7 shows an isometric view of one embodiment of a tent barrier;

FIG. 8 shows a top view of a side wall canopy of the tent of FIG. 7;

FIG. 9 shows a plan view of a canopy body of one embodiment of the disclosure;

FIG. 10 shows a profile of the barrier tent;

FIG. 11 shows a top view of a suction tube of one embodiment of the disclosure;

FIG. 12 shows a side view of a connector of one embodiment of the disclosure.

### DETAILED DESCRIPTION OF THE EMBODIMENTS

Disclosed in some forms is a barrier tent comprising: a canopy, at least a portion of the canopy being composed of flexible sheet material; a frame for supporting the canopy; the canopy and frame being configured such that an internal surface of the canopy defines at least a portion of a patient segregation cavity to enclose a patient's head and at least a portion of the patient's body within the patient segregation cavity; a gas input extending through the canopy, the gas input being positioned at a location configured such that supply of a gas through the input limits movement of aerosolised particles generated by or adjacent the patient toward the internal surface of the canopy.

In some forms, the frame comprises a plurality of flexible elongate members.

In some forms the segregation cavity has an upper portion or an apex and in some forms the gas input is located in or adjacent the upper portion or apex.

In some forms the input comprises a full cone spray nozzle.

In some forms supply of a gas through the input disperses gas downwards within the segregation cavity in a conical spray pattern.

In some forms the tent further comprises one or more semi-sealed apertures extending through the cavity to allow access to the patient. In some forms the one or more semi-sealed apertures comprise an aperture with a flap or other sheet material covering the aperture.

In some forms the barrier tent has suction assisted removal of gases within the segregated patient cavity.

In some forms suction is assisted through suction elements located at the base of the tent. In some forms the suction elements are perforated. In some forms the suction elements are in the form of strips.

In some forms an impermeable base sheet extends across the bottom of the barrier tent.

In some forms the tent includes an attachment strip or an adhesive to removably connect the tent to a bed.

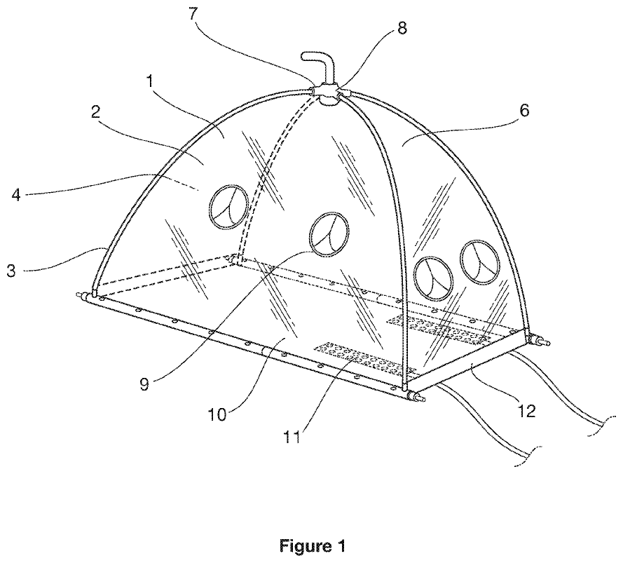

Referring now to FIG. 1, disclosed is a barrier tent 1 comprising a canopy 2 and frame 3 defining an interior segregation cavity 4. The interior segregation cavity is sized and shaped to allow a portion of a patient for example, a patient's head and torso to be positioned therein. In some forms the barrier tent is also sized and shaped to fit on a standard hospital bed.

The frame 3 of the barrier tent comprises a plurality of elongate shafts 5 that are flexible and connect together to form a frame that supports the canopy 2. In the illustrated form the shafts 5 are oriented orthogonal to one another and cross over at a central portion at or proximal to an apex of the tent 7. The shafts 5 curve from a lower corner to the apex 7. In the illustrated form the curve of the shafts 5 steepens toward the corner of the barrier tent. The frame is designed to define an interior space with an upper portion 6 that is located near and around the apex.

An input 8 is located at or near the apex or in the upper portion of the interior cavity. The input 8 may be in the form of an opening or aperture and may be formed of plastic metal or other shapeable or mouldable material that allows for a channel or aperture to be formed allowing input of gas therein.

Access ports 9 are located in the canopy and extending into the interior space. The access ports in this form are circular apertures with covers that allow entry through breaks in the cover. The ports 9 are located intermediate the upper and lower portion of the segregation cavity 4 or in an upper portion of the wall such that the ports are located away from movement of aerosol material within the cavity. In the illustrated form, access ports are located at two sides of the barrier tent although they may be located at more sides or in fewer sides. In the illustrated form the access ports are a pair of access ports spaced apart from one another such that a user can insert an arm into each access port providing for two-handed work. The access ports may be specifically located in an upper portion of the barrier tent.

A base 10 may extend across the lower portion of the tent. The base may be in the form of a flexible sheet. One or more flat suction strips 11 may be located in the interior of the cavity 4 and may run across the base. In the illustrated form the flat suction strips may comprise flat perforated strips running across a portion of the base of the tent.

An attachment strip 12 which may be adhesive or weighted, may be located at the bottom of the tent to connect with bedding or a surface.

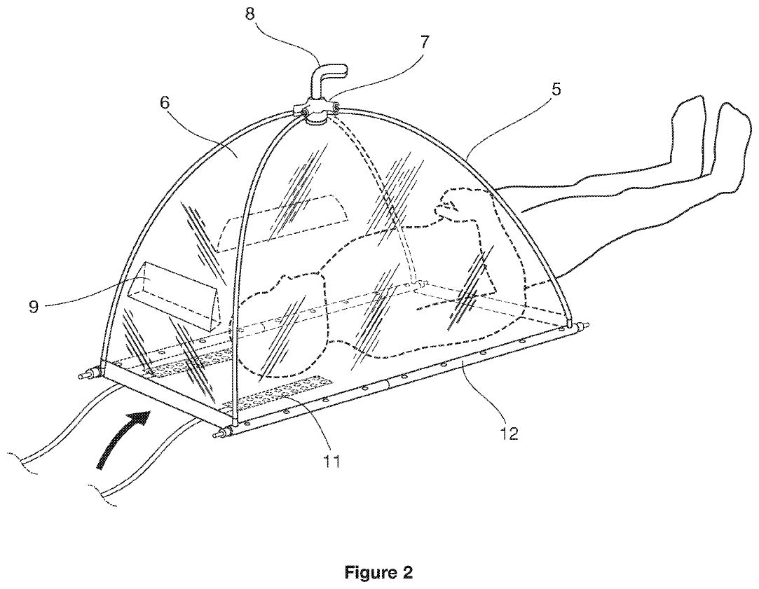

FIG. 2 shows a second embodiment of the barrier tent in which the ports 9 are in the form of flaps extending into the segregation cavity. In this illustrated form, disclosed is a barrier tent 1 comprising a canopy 2 and frame 3 defining an interior segregation cavity 4.

The frame 3 of the barrier tent comprises a plurality of elongate shafts 5 that are flexible and connect together to form a frame that supports the canopy 2. In the illustrated form the shafts 5 are shaped as a portion of a circle, are oriented orthogonal to one another and cross over at a central portion at or proximal to an apex of the tent 7. The frame is designed to define an interior space with an upper portion 6.

An input 8 is located at or near the apex or in the upper portion of the interior cavity. The input 8 may be in the form of an opening or aperture and may be formed of plastic metal or other shapeable or mouldable material that allows for a channel or aperture to be formed allowing input of gas therein. In the illustrated form the input 8 may provide a full cone spray from the apex into the interior cavity.

Access ports 9 are located in the canopy and extend into the interior space. The access ports in this form are rectangular apertures with covers in the form of flaps that are attached at the top of the aperture. The flaps allow entry through breaks in the cover. The ports 9 are located intermediate the upper and lower portion of the segregation cavity 4 or in an upper portion of the wall such that the ports are located away from movement of aerosol material within the cavity. In the illustrated form, access ports are located at two sides of the barrier tent although they may be located at more sides or in fewer sides. In the illustrated form the access ports are a pair of access ports spaced apart from one another such that a user can insert an arm into each access port providing for two-handed work. The access ports may be specifically located in an upper portion of the barrier tent.

A base 10 may extend across the lower portion of the tent. The base may be in the form of a flexible sheet. In some forms the diameter of the base is approximately 575-590 mm. One or more flat suction strips 11 may be located in the interior of the cavity 4 and may run across the base. In the illustrated form the flat suction strips may comprise flat perforated strips running across a portion of the base of the tent.

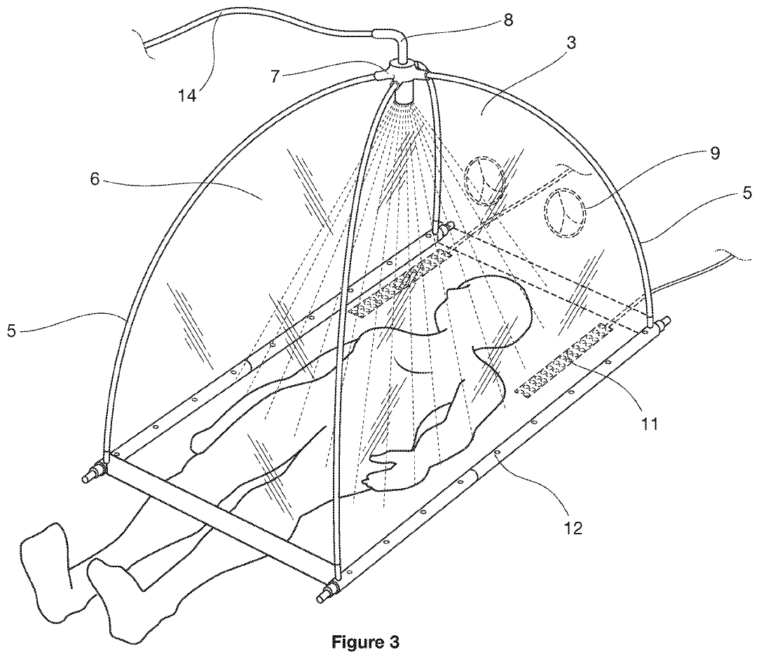

Referring now to FIG. 3, a barrier tent 1 is shown in which the canopy 3 includes two access ports 9 located at the head end of the barrier tent. In this form the flexible elongate shafts 5 again curve over the patients and cross over where they meet at an apex 7. An input 8 for supply of gas is positioned at the apex 7 or adjacent the apex such that it is positioned to deliver gas to an upper portion 6 of the interior cavity.

The input 8 provides a cone spray into the segregation cavity. The gas supplied to the tent may be air or oxygen.

The embodiment shows suction strips 11 which may be any length or width and may be perforated and flat for suction of gases from the interior cavity. In alternative forms the suction elements may be plates or tubes which are perforated and allow for suction of gases from the lower portion of the interior cavity.

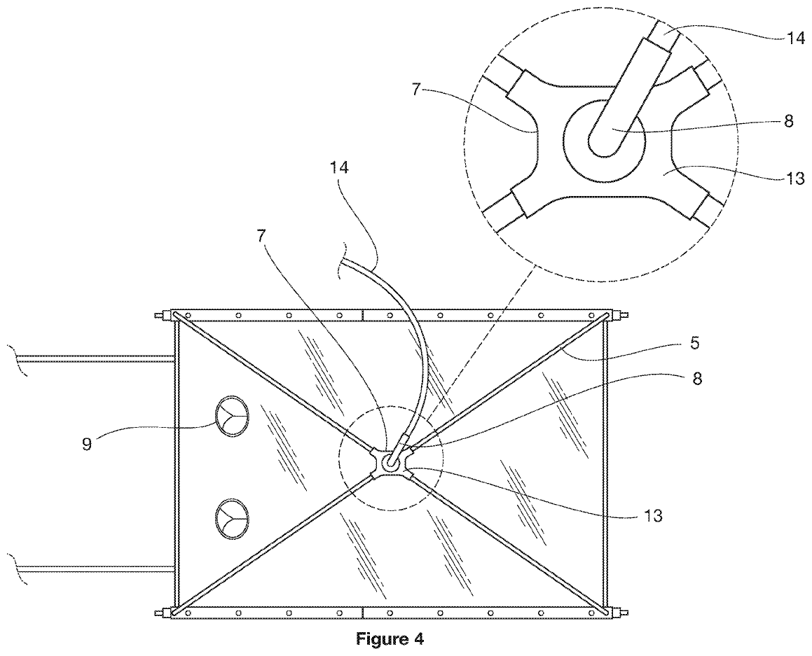

As seen in FIG. 4, an upper view of the tent shows the frame shafts 5 meeting and crossing over one another at the apex 7. The apex incorporates an input 8 for input of gas into the interior cavity. Tubing 14 is connected with the input 8 and extends therefrom to supply gas to the interior cavity of the tent. In the illustrated form the input 8 is attached with an apex support 13 which is located at the apex 7. The apex support comprises attachment for each shaft 5 and allows them to meet centrally at the apex 7 while also including the input 8 to which the tubing or other channel is attached for delivery of gas to the interior cavity of the barrier tent.

The input 8 is shown in more detail in the detail section of FIG. 4. The input 8 may comprise a tube attachment portion and a prong for extending through the canopy into the interior cavity. In some forms the input forms part of a connector for the frame, in some forms the input is separate from the frame and is engaged with the frame. In some forms the input includes an elbow such that the tubing meets the input at a longitudinal portion of the input and the input turns through an angle and extends into the interior cavity of the tent. The input 8 is configured such that an incoming flow of gas is directed downward onto the patient. The downward movement creates a wall-jet vortex at the side wall meaning that droplets from secondary atomisation at the patient are directed away from the walls of the barrier tent reducing the risk of infection.

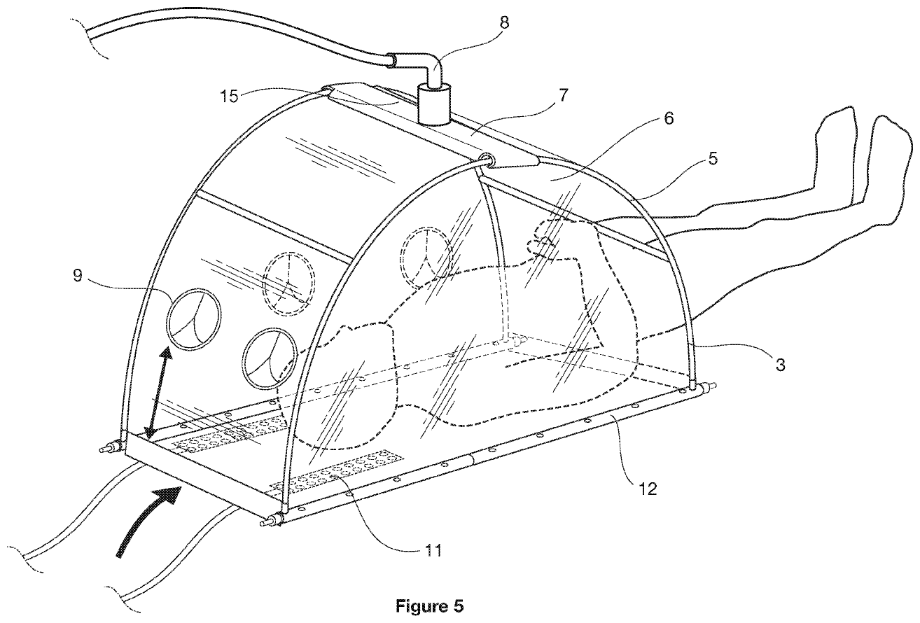

FIG. 5 shows a further embodiment of the tent barrier 1. In this embodiment the frame forms an arch shape extending over the patient and the canopy defines an interior space which can enclose the patient's head and body. The frame members 5 extend parallel to one another to form a curved arch. The frame further includes an upper bar 15 that extends between the frame members 5 at an upper portion 6 of the interior cavity. The input 8 for input of gas is located on the upper bar 15.

Access ports 9 are located in the canopy and extending into the interior space. The access ports in this form are circular apertures with covers that allow entry through breaks in the cover. The ports 9 are located intermediate the upper and lower portion of the segregation cavity 4 or in an upper portion of the wall such that the ports are located away from movement of aerosol material within the cavity. In the illustrated form, access ports are located at a curved end of the barrier tent adjacent a patient's head (in use) although they may be located at more sides or in fewer sides. In the illustrated form the access ports are a pair of access ports spaced apart from one another such that a user can insert an arm into each access port providing for two-handed work.

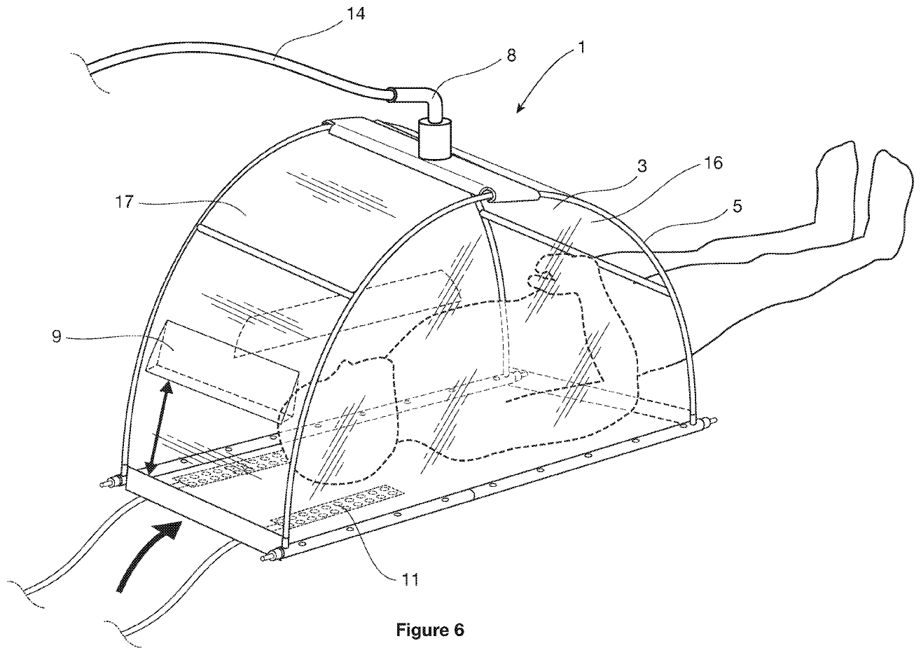

FIG. 6 shows a further embodiment of the tent similar to FIG. 5. In this form access ports 9 are in the form of a single rectangular aperture with a cover in the form of a flap that is attached at the top of the aperture. The port 9 is located intermediate the upper and lower portion of the segregation cavity 4 or in an upper portion of the wall such that the ports are located away from movement of aerosol material within the cavity.

In the embodiment shown in FIGS. 5 and 6 the input is located at or near the upper bar 15 of the frame. In some forms the upper bar of the frame includes an elbow joint that comprises the input 8 and allows gas to be delivered.

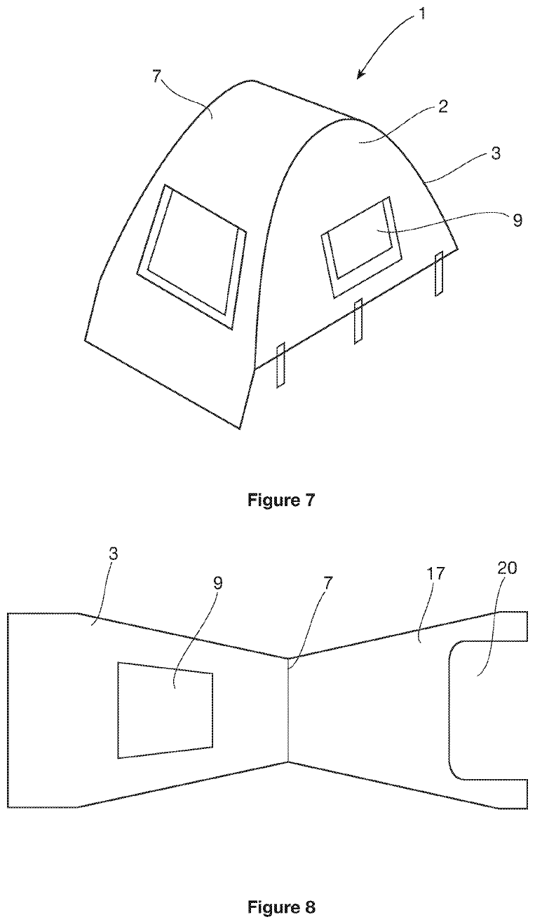

Referring now to FIG. 7, disclosed is a barrier tent 1 comprising a canopy 2 and frame 3 defining an interior segregation cavity 4. The frame may be integral with the canopy or separate from the canopy. The interior segregation cavity is sized and shaped to allow a portion of a patient for example, a patient's head and torso to be positioned therein. In some forms the barrier tent is also sized and shaped to fit on a standard hospital bed.

In the illustrated form the frame 3 forms a curved external wall having two spaced apart sides and a central wall curving upwardly over the sides to a peak 7 which extends across the width of the tent.

In this form access ports 9 are in the form of two rectangular apertures with covers in the form of flaps 14 covering the apertures and attached to the canopy at an upper end of the aperture. The ports 9 are located at the end and side of the barrier tent to allow access.

FIG. 8 shows a plan view of the curved wall of the barrier tent spread out before formation if the tent. The curved wall tapers to the upper portion which will form the peak of the barrier tent. In addition, the curved wall includes an opening 20 at the foot end of the tent to allow a patient's body and/or legs to extend from the tent. This opening will be partly or fully closed by the body of a patient who is in the tent.

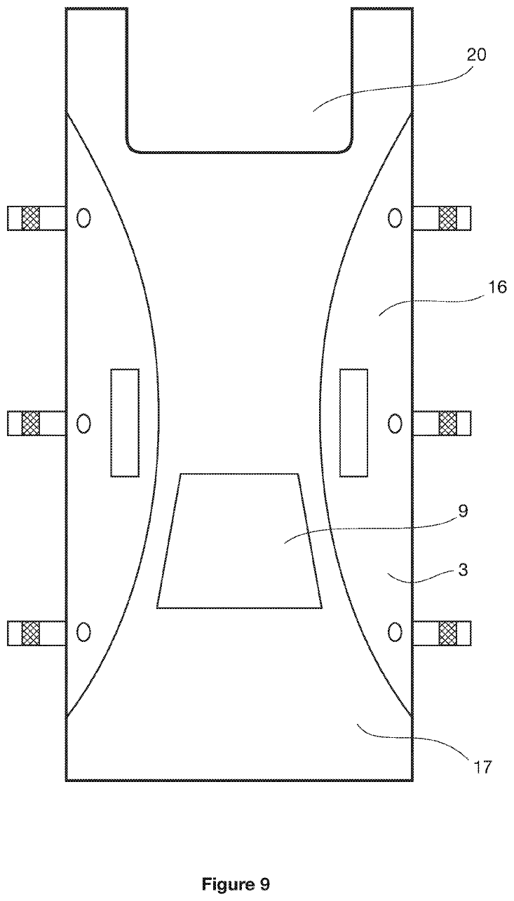

FIG. 9 shows an alternative shape of the barrier tent curved wall. The shape includes side walls 16 and a top curved wall 17 that turn through an angle with respect to one another in use. The wall further includes an opening 20 for the patient's body or legs to extend through. Again an attachment strip 12 may be adhesive or weighted, may be located at the bottom of the tent to connect with bedding or a surface.

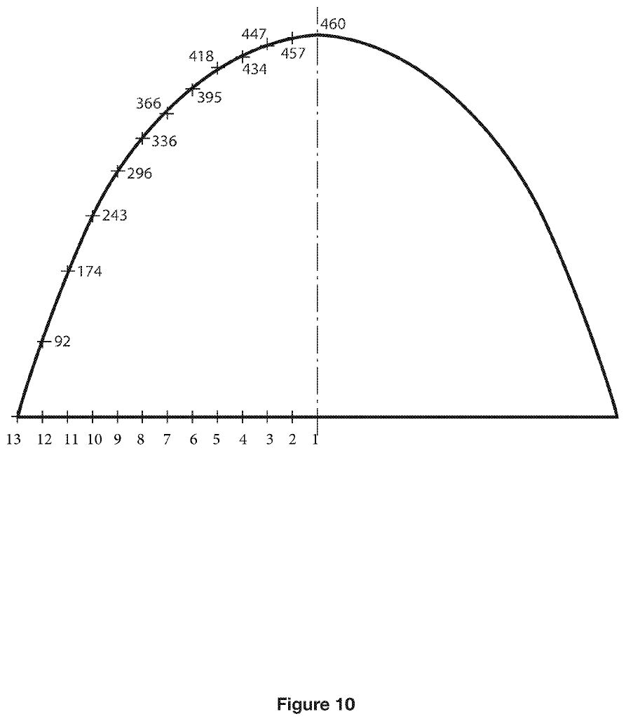

Referring now to FIG. 10, the cross-sectional profile of the interior cavity of the barrier tent is shown. The profile curves steeply from the base and tapers to the peak of the tent.

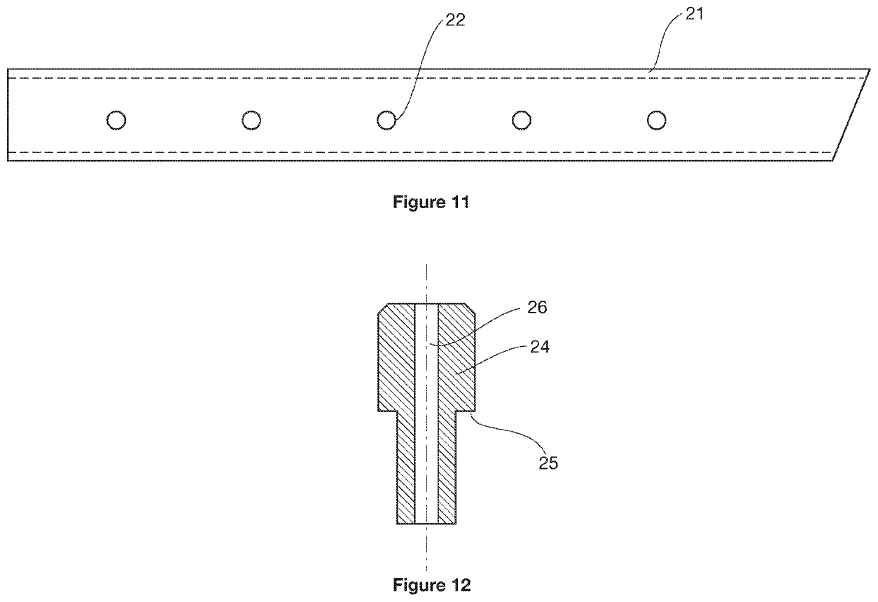

FIG. 11 shows one embodiment of a suction tube 21 which is configured to be fitted at a lower section of the barrier tent 1. The suction tube 21 has a circular cross section and includes a plurality of perforations 22 to allow for suction from the barrier tent at a lower end of the tent. In some forms the flexible shaft of the tent may be connected with and extend from the suction tube through an aperture in the tube.

In use, suction tubes 21 are extended along either side of the lower area of the barrier tent. The barrier tent is secured to the suction tubes by Velcro or other attachment. At least a portion of the suction tubes are covered by the base of the tent. Operating room wall or portable suction is attached to the suction tubes and a filter is located between the suction tubes and the wall or portable suction.

FIG. 12 shows an example of a connector that may be utilised as the input allowing gas into the interior cavity of the barrier tent. The connector comprises a body 24 having a flange 25 and an interior channel 26 which allows for delivery of gas from a tube to the interior cavity of the tent.

The barrier tent provides flexibility in protecting health workers from aerosolised material emanating from the patient through a combination of a physical barrier and the flow of gas from an upper portion of the tent which may deflect movement of the aerosolised material to limit the material contacting the interior of the tent particularly in the upper portions of the tent. Flow of gas within the interior of the barrier tent is managed by the delivery of gas in some forms through a full cone spray nozzle, the delivery being from an upper portion of the interior of the tent and directed downwardly. In addition, the suction tubes from a lower region promote the movement of gas that limits infection.

While the technology has been described in reference to its preferred embodiments, it is to be understood that the words which have been used are words of description rather than limitation and that changes may be made without departing from its scope as defined by the appended claims.

It is to be understood that a reference herein to a prior art document does not constitute an admission that the document forms part of the common general knowledge in the art in Australia or in any other country.

In the claims which follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the technology.

1. A barrier tent comprising:
a canopy, at least a portion of the canopy being composed of flexible sheet material;
a frame for supporting the canopy;
the canopy and frame being configured such that an internal surface of the canopy defines at least a portion of a patient segregation cavity to enclose a patient's head and at least a portion of the patient's body within the patient segregation cavity;
a gas input extending through the canopy, the gas input being positioned at a location configured such that supply of a gas through the input limits movement of aerosolised particles generated by or adjacent the patient toward the internal surface of the canopy.

2. A barrier tent as defined in claim 1, wherein the frame comprises a plurality of flexible elongate members.

3. A barrier tent as defined in claim 1, wherein the segregation cavity has an upper portion or an apex.

4. A barrier tent as defined in claim 3, wherein the gas input is located in or adjacent the upper portion or apex.

5. A barrier tent as defined in claim 1, where the input comprises a full cone spray nozzle.

6. A barrier tent as defined in claim 1 wherein supply of a gas through the input disperses gas downwards within the segregation cavity in a conical spray pattern.

7. A barrier tent as defined in claim 1 further comprising one or more semi-sealed apertures extending through the cavity to allow access to the patient.

8. A barrier tent as defined in claim 7, wherein the one or more semi-sealed apertures comprise an aperture with a flap or other sheet material covering the aperture.

9. A barrier tent as defined in claim 1, further comprising suction assisted removal of gases within the segregated patient cavity.

10. A barrier tent as defined in claim 9, wherein suction is assisted through suction elements located at the base of the tent.

11. A barrier tent as defined in claim 10 wherein the suction elements are perforated.

12. A barrier tent as defined in claim 10 or 11, wherein the suction elements are in the form of strips or tubes extending along a portion of the base.

13. A barrier tent as defined in claim 1, further including a impermeable base sheet extending across the bottom of the barrier tent.

14. A barrier tent as defined in claim 1 further comprising an adhesive to removably connect the tent to a bed.
